(19) United States
(12) Patent Application Publication
(10) Pub. No.: US 2025/0041469 A1
(43) Pub. Date: Feb. 6, 2025

(54) FOG PRODUCTION UNIT FOR A VEHICLE, IN PARTICULAR A TRUCK

(71) Applicant: Frans Veugen Bedrijfshygiëne B.V., Nederweert (NL)

(72) Inventors: Franciscus Jacobus Petrus Paulus Veugen, Nederweert (NL); Paul Martin Veugen, Nederweert (NL); Rob Andries Veugen, Nederweert (NL)

(21) Appl. No.: 18/836,816

(22) PCT Filed: Feb. 16, 2023
(86) PCT No.: PCT/NL2023/050072

§ 371 Date: Aug. 8, 2024

(30) Foreign Application Priority Data
Feb. 16, 2022 (NL) 2030961

(51) Int. Cl. A61L 2/22; A61L 2/24; A61L 2/26

(52) U.S. Cl. CPC A61L 2/22; A61L 2/24; A61L 2/26; A61L 2202/14; A61L 2202/15; A61L 2202/16

(57) ABSTRACT

A fog production unit for a truck having a pressurized air storage tank, includes a container for holding a liquid fogging medium, a fogging nozzle in fluid connection with the container via a supply, a control system and a fluid delivery system, such as a pump or a venturi nozzle, configured to in operation deliver the liquid fogging medium from the container to the fogging nozzle, the nozzle further connected to an air supply which is configured to be connected to the pressurized air storage tank of the vehicle such that the liquid fogging medium is in operation exposed to the pressurized air of the pressurized air storage vehicle tank of the vehicle at the fogging nozzle and wherein the air supply is controllably supplied in response to a control signal of the control system.

### TECHNICAL FIELD

The present disclosure relates to a fog production unit for a vehicle, in particular a truck. The invention also pertains to a vehicle comprising such fog production unit and to a method for installing such fog production unit.

### BACKGROUND ART

Vehicles need regular conditioning, cleaning and/or disinfecting in order to safely use them. This is even more important in case of touring cars and in particular cargo trucks. It is known to disinfect and/or condition such vehicles by using a fog production unit for humidity conditioning and/or to disinfect by adding a disinfectant to the fogging medium. A fog production device of this kind is described in United States patent U.S. Pat. No. 9,907,877. The prior art device forces a pressurized air stream through a nozzle, at which nozzle the pressurized air is brought into contact with a liquid which is thereby atomized. This way of producing a mist or fog is as such known in the art in several ways. Such fog production unit is either a portable fog production unit that is placed inside of the cargo space of the vehicle at certain cleaning moments, or it could be an integrated fog production unit that is mounted to the vehicle itself and carried along. The latter is in many cases undesired as a bulky fog production unit takes up space that could otherwise be used for transporting cargo, and besides the added weight adds up to fuel usage of such vehicle.

### SUMMARY OF THE INVENTION

In a first aspect of the present invention, the fog production unit for a vehicle, in particular a truck, the vehicle comprises a pressurized air storage tank, the fog production unit comprising a container for holding a liquid fogging medium, a fogging nozzle in fluid connection with the container by means of a supply, a control system and a fluid delivery system, such as a pump or a venturi nozzle, configured to in operation deliver the liquid fogging medium from the container to the fogging nozzle, characterized in that the nozzle is further connected to an air supply, wherein the air supply is configured to be connected to the pressurized air storage tank of the vehicle such that the liquid fogging medium is in operation exposed to the pressurized air of the pressurized air storage tank of the vehicle at the fogging nozzle and wherein the air supply is controllably supplied in response to a control signal of the control system.

The fog production unit according to the invention is not relying on a compressor for pressurized air as is the case in prior art implementations. The pressurized air storage tank of the vehicle itself, which is normally used to power e.g. pneumatic brakes and other pneumatic systems of the vehicle, is usually provided with pressurized air by means of a compressor that is powered by the vehicle itself, e.g. by means of a drive shaft powered by the power unit of the vehicle. By providing a fog production unit that provides an air supply connection to the pressurized air tank of the vehicle itself as a supply of pressurized air for the fog production unit in the cargo space, the fog production unit according to the present invention can be executed less bulky and significantly lighter.

The fog production unit comprises a container for holding a liquid fogging medium. Such container can be e.g. a tank, jerry can or other liquid holding compartment. It comprises a liquid supply out of the container such as a hose or other transport means and usually also comprises a refill opening to refill the liquid, although in some applications such refill opening may not be provided, e.g. in case the container is provided as a “cartridge” which is filled at the manufacturing location. The latter may be advantageous in case of chemicals or in case the a certain mix of liquids is needed in a very specific ratio.

Further a fogging nozzle is provided. There are several suitable nozzles for this type of applications of fog production by pressurized air. Suitable nozzles include e.g. dry mist nozzles wherein a supply of compressed air is fed along a liquid supply restriction and wherein usually an air reflector is mounted downstream the supply restriction, in order to create a shockwave between the reflected air and the freshly supplied air. This shockwave separates the drops of supplied liquid into smaller drops in order to produce a fog or mist at the exit of the fogging nozzle.

A fluid delivery system, such as a pump or other dosing and supplying system is provided in order to provide the fogging medium liquid from the container to the nozzle. Other dosing and supplying systems include self-priming systems such as e.g. a venturi nozzle which may be integrated into the fogging nozzle, or placed separately in the supply line and is configured to in operation deliver the liquid fogging medium from the container to the fogging nozzle. By forcing pressurized air through such venturi nozzle, the liquid is sucked into the supply line to the nozzle. In embodiments where the liquid container is stored above the nozzle height, such fluid delivery system may also be implemented as a controllable valve wherein the liquid delivery is self-primed by means of gravitational influences. Such liquid is usually water(based) but other liquids can also be suitable for specific applications.

The air and liquid supplies to the nozzle are controlled by a control system. Such control system controls the supplies by means of suitable control means such as e.g. controlled valves or may alternatively control the pump of the liquid supply directly without a valve. Such controlled actuators may be connected to the control system in a direct physical fashion, wireless or in any other suitable way such that the control system can influence the state and/or operation of the actuators. The control system may be implemented as a single physical unit, such as a physical control box comprising electronics, but may also be implemented as a distributed system, wherein several control components are placed in various locations in or on the vehicle.

Electrical power of the components may be provided for by a dedicated electrical power supply such as a battery or may be supplied for by the electrical power supply of the vehicle itself.

In an embodiment of the fog production unit for a vehicle, the control system comprises a data logger, the data logger comprising storage means for storing data relating to the operation of the for production unit. Disinfection operations and environmental conditions inside the cargo space can have significant impact on quality and safety of the transported cargo. For example, transport of livestock can spread deceases amongst transported batches if not disinfected correctly after each batch, and quality of fruits and vegetables can be highly influenced if the humidity conditions and/or dry mist conditions inside the cargo space are not kept at optimal conditions during transport. Clients of transporters are increasingly involved in conditions during and in between transports and demand optimal conditions during and certified and traceable cleaning procedures in between each transport. Therefore a logging of these conditions and environmental parameters is increasingly important. As the present invention enables cleaning and conditioning while driving because of the integration of the fog production unit with the vehicle, this logging becomes even more important, as cleaning and conditioning are now outside of the view of the driver and/or customers. Storing data may be implemented as a printer, but may also be implemented as writing on a storage medium such as a disk drive, flash drive or other suitable and in itself known ways. The unit may also implement read out means for reading the stored information by means of mobile devices, a print out, or remote readout in a remote software client.

In a further embodiment of the fog production unit for a vehicle, the data logger is configured to write time stamped information relating to at least one of the group consisting of activating the fog production unit, duration of the fog production operation, and amount of consumed liquid fogging medium. By registering events like activation of the fog production unit, ending of the operation, error messages, amount of consumed liquid fogging medium and/or duration of the operation, this information can be observed by users either locally on the unit or remotely by means of digital communication means which are as such commonly known in the art.

In an embodiment of the fog production unit for a vehicle, the fog production unit further comprising a disinfection container for holding disinfectant liquid. In many practical applications, the fog from the fog production unit is used for disinfection purposes. The disinfecting agent may be premixed in the container by manufacturer or by an operator, but it may be advantageous to supply two separate containers for holding the main fogging medium liquid, e.g. water, and the disinfectant liquid in separate containers. By supplying them in separate containers the mix can be made on the fly and further the mix could even be adapted based on certain conditions, for example loading conditions, (outside) temperature, humidity or type of cargo load.

In a further embodiment of the fog production unit for a vehicle, the disinfectant liquid is pumpable by means of a pump to a mixing means, wherein the mixing means are configured to receive the fogging medium and the disinfectant liquid and to provide the mixed liquid to the fogging nozzle. The mixing means can be implemented as a relatively simple Y- or T-shaped tubing piece, wherein a stream of fogging medium such as water is supplied and another stream of disinfectant agent is supplied, but may also be implemented with an active mixing component, for example by a rotor in a buffer container, by a cyclone in the tubing or any suitable mixer.

In a further embodiment of the fog production unit for a vehicle, the fog production unit further comprises a dosing control means for controlling the amount of disinfectant liquid in relation to the amount of fogging medium. In some applications the ratio of the mix between fogging medium (such as water) and the disinfectant liquid is important for a correct disinfection operation. In those cases the fog production unit may comprise dosing control means. Such dosing control means may comprise separate pumps for both containers, or a single pump in combination with a controlled valve to control the amount of liquid from each container.

In an embodiment of the fog production unit for a vehicle, it further comprises activation means to activate the fog production unit. Activation means may comprise an automated activation by means that activates the operation at certain intervals or at certain events such as after loading or unloading, but could also be triggered by environmental parameters sensed such as humidity, temperature, detection of pollution or other events. Alternatively or in addition the activation means may comprise manual activation means such as buttons, switches and the like. Alternatively or in addition the activation means may also be implemented remotely by means of communication means, such that a fleet of multiple vehicles may be activated concurrently such that a fleet of multiple vehicles can be controlled by a central controller e.g. at the transporters head quarters.

In a further embodiment of the fog production unit for a vehicle, the activation button is implemented as a wireless button which is wirelessly connected to an associated receiver, such that in operation the receiver is configured to receive an activation signal from the wireless button. The fog production unit can be operated during driving the vehicle and/or when stationary. A button in the driver cabin can therefore be advantageous in order to activate the system while driving. The operator of the vehicle is also able to choose the right timing of the operation or able to postpone such operation, for example in case the pressure of the pressurized air is low due to other pneumatic operations. Such button in the drive cabin may be implemented wired or wireless. Wireless communication is implemented by means of in itself known communications. In case of wireless communication the associated receiver is part of the control system such that pushing the button provides an activation signal to the control system.

In a second aspect of the present invention, a vehicle is provided in particular a truck, comprising a power unit for driving the vehicle, a driver cabin comprising control means for controlling the acceleration, braking and steering of the vehicle and a cargo space, further comprising a pressurized air storage tank, a compressor for supplying compressed air to the pressurized air storage tank, characterized in that the vehicle comprises a fog production unit according to any one of preceding claims, and wherein the fog production unit is connected to the pressurized air storage tank and wherein the fogging nozzle of the fog production unit is mounted inside the cargo space. The use of the compressor of the vehicle itself contributes to a lighter and less complex fog production system, which contributes to less fuel usage by the vehicle and a reduction of used cargo volume by the fog production system as a whole which can be used to transport cargo.

In an embodiment of the vehicle, the compressor is mechanically driven by the power unit of the vehicle. The vehicle comprises a compressor used to pressurize the pressurized air storage tank. The pressurized air in the pressurized air storage tank may be used for pneumatic operations such as brakes, loading lifts and the air supply to the fog production unit. The compressor is mechanically driven by the power unit of the vehicle, either directly by a drive shaft between the power unit and the compressor, or indirectly by e.g. an axle connected to the wheels, or by means of a belt, chain or other transmission means. The compressor may also be electrically driven by the electrical power (supply) means of the vehicle in case of electric or hybrid vehicles. The vehicle may comprise of a driven part (tractor) and a separate trailer connected to the driven part, in such cases the compressor and/or compressed air storage tank may be mounted on the tractor or on the trailer part of the vehicle.

In an embodiment of the vehicle, sensors are provided for measuring environmental parameters within the cargo space and configured such that in operation the sensors provide sensor information to the data logger of the fog production unit. The environmental parameters may comprise temperature of the cargo space, humidity of the cargo space, the open and/or closed state of the cargo space, and/or luminosity within the cargo space.

In an embodiment of the vehicle, activation means are provided to activate the fog production unit are mounted in the driver cabin of the vehicle. Activation means may comprise an automated activation by means that activates the operation at certain intervals or at certain events such as after loading or unloading, but could also be triggered by environmental parameters sensed such as humidity, temperature, detection of pollution or other events. Alternatively or in addition the activation means may comprise manual activation means such as buttons, switches and the like. Alternatively or in addition the activation means may also be implemented remotely by means of communication means, such that a fleet of multiple vehicles may be activated concurrently such that a fleet of multiple vehicles can be controlled by a central controller e.g. at the transporters head quarters. The activation button may be implemented as a wireless button which is wirelessly connected to an associated receiver, such that in operation the receiver is configured to receive an activation signal from the wireless button. The fog production unit can be operated during driving the vehicle and/or when stationary. A button in the driver cabin can therefore be advantageous in order to activate the system while driving. The operator of the vehicle is also able to choose the right timing of the operation or able to postpone such operation, for example in case the pressure of the pressurized air is low due to other pneumatic operations. Such button in the drive cabin may be implemented wired or wireless. Wireless communication is implemented by means of in itself known communications. In case of wireless communication the associated receiver is part of the control system such that pushing the button provides an activation signal to the control system.

In a third aspect of the present invention, a method for installing a fog production unit in a vehicle, in particular a truck, is provided, comprising the steps of:
mounting the fog production unit in the vehicle, wherein the fogging nozzle is mounted within the cargo space;
   connecting the air supply of the fog production unit to the pressurized air storage tank of the vehicle;
   mounting an activation means to activate the fog production unit in the driver cabin.

Such installation of the fog production unit results in a lighter and less complex implementation of the fog production system, while keeping the same or even improved operation thereof.

### BRIEF DESCRIPTION OF THE DRAWINGS

Further scope of applicability of the present invention will become apparent from the detailed description given hereinafter. However, it should be understood that the detailed description and specific examples, while indicating embodiments of the invention, are given by way of illustration only, since various changes and modifications within the scope of the invention will become apparent to those skilled in the art from this detailed description and the accompanying schematic drawings which are not limitative of the present invention, and wherein:

FIG. 1 is a schematic perspective view of a vehicle with a fog production unit according to the present invention;

FIG. 2 is a schematic perspective view of fog production unit according to the present invention;

### DETAILED DESCRIPTION OF EMBODIMENTS

The present invention will now be described with reference to the accompanying drawings, wherein the same reference numerals have been used to identify the same or similar elements throughout the several views.

It is noted that the drawings are schematic, not necessarily to scale and that details that are not required for understanding the present invention may have been omitted. The terms “upward”, “downward”, “below”, “above”, and the like relate to the embodiments as oriented in the drawings, unless otherwise specified. Further, elements that are at least substantially identical or that perform an at least substantially identical function are denoted by the same numeral, where helpful individualized with apostrophe suffixes.

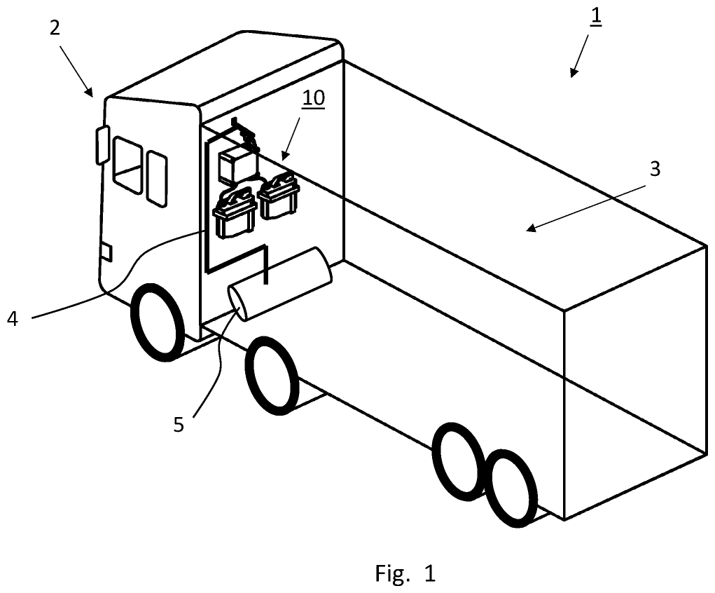

FIG. 1 schematically illustrates a perspective view of an example of a vehicle 1, in particular a truck, comprising a power unit (not shown) for driving the vehicle 1, a driver cabin 2 comprising control means (not shown) for controlling the acceleration, braking and steering of the vehicle and a cargo space 3, further comprising a pressurized air storage tank 5, a compressor (not shown) for supplying compressed air to the pressurized air storage tank 5. The vehicle comprises a fog production unit 10 wherein the fog production unit 10 is connected to the pressurized air storage tank 5 by means of pressurized air hose 4. The fogging nozzle of the fog production unit 10 is mounted inside the cargo space 3.

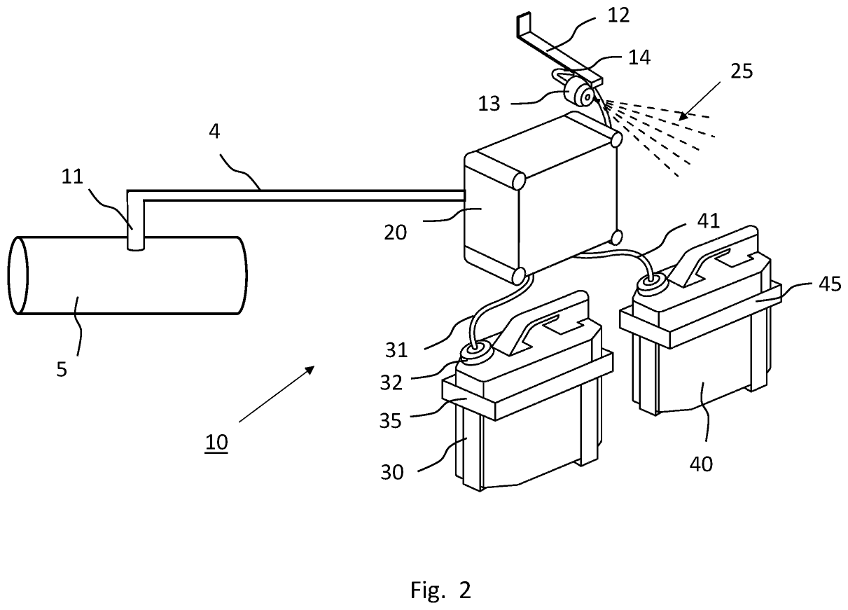

FIG. 2 is a schematic perspective view of an example of a fog production unit 10 according to the present invention. The pressurized air storage tank 5 is filled with pressurized air by means of a compressor that is integral part of the vehicle. The pressurized air from the pressurized air storage tank is used for several pneumatic operations of the vehicle such as pneumatic brakes and the loading bay lift. A pressurized air hose 4 is connected via the control box to an outlet of the pressurized air storage tank 5 by means of a quick release connector 11, in casu a ½″ connection thread Festo connector. The hose 4 provides the pressured air from the tank 5 to the nozzle 13 which is mounted inside of the cargo space 3 of the vehicle 1 by means of mounting bracket 12. The air hose is fed through the control box such that the control system is able to close the infeed of air to the nozzle by means of a controllable valve. Further are measurement means provided to measure the pressure of the air supply before it is fed to the nozzle. In case the pressure measurement indicates an air pressure under a predetermined threshold, the air supply is closed and the event is logged in order to guarantee a safe operation of the device. The valve of the pressurized air hose, also comprises a pressure reduce function, in order to reduce the pressure in case the supplied pressure is too high. It will be appreciated that in an alternative embodiment, the air hose does not physically pass through the control box but comprises remotely controllable (reduce) valves and remotely readable pressure measurement means, such that the pressurized air hose can be fed in an efficient way directly to the nozzle, while the control box is still able to control and/or stop the supply of air to the nozzle.

The nozzle 13 is further provided with liquid supply hose 14 in order to eject a fog or mist 25 at its exit. Control box 20 comprises electronics to control fluid pumps (not shown) which are placed inside box 20. The pumps dose amounts of fogging medium, in casu demineralized water, via liquid supply hose 31 from liquid fogging medium holding container 30. The container 30 is mounted to the back wall of the cargo space 3 by means of a mounting bracket 35 and comprises a refill opening 32 to refill the liquid between operations. The control box 20 further comprises a dosing pump (not shown) to dose an amount of disinfectant liquid via supply hose 41 from disinfectant holding container 40, which is mounted to the back wall of the cargo space 3 by means of mounting brackets 45. It will be appreciated that the containers 30, 40 may alternatively be mounted in a side compartment of a trailer for easy access and in order to take up less cargo volume inside of the cargo space 3. The driver cabin 2 of the vehicle comprises a wireless activation button (not shown) that is connected to an associated receiver in control box 20. Activating the button triggers the activation of the fog production unit in the cargo space 3. This may be activated during stationary state of the vehicle or during driving the vehicle, e.g. after unloading the cargo.

Further, the terms and phrases used herein are not intended to be limiting; but rather, to provide an understandable description of the invention. The terms “a” or “an”, as used herein, are defined as one or more than one. The term “plurality”, as used herein, is defined as two or more than two. A plurality may also indicate a subset of two or more, out of a larger multitude of items. The term “another”, as used herein, is defined as at least a second or more. The terms including and/or having, as used herein, are defined as comprising (i.e., open language). The term “coupled”, as used herein, is defined as connected, although not necessarily directly.

Terms “front (panel)”, “back (panel)”, “top (panel)” and “bottom (panel)” are not intended to restrict the possible orientations of the product in view of the user, but merely to clearly define and describe the interrelationships between the plurality of panels.

Elements and aspects discussed for or in relation with a particular embodiment may be suitably combined with elements and aspects of other embodiments, unless explicitly stated otherwise. The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A fog production unit for a vehicle of a type including a pressurized air storage tank, the fog production unit comprising:
a container for holding a liquid fogging medium,
a fogging nozzle in fluid connection with the container,
a control system,
a fluid delivery system configured to, in operation, deliver the liquid fogging medium from the container to the fogging nozzle,
an air supply for connecting the nozzle to the pressurized air storage tank of the vehicle such that the liquid fogging medium is, in operation, exposed to the pressurized air of the pressurized air storage tank of the vehicle at the fogging nozzle and
a control system for providing a control signal to control the air supply.

2. A fog production unit for a vehicle according to claim 1, wherein the control system comprises a data logger, the data logger comprising a storage device for storing data relating to the operation of the fog production unit.

3. A fog production unit for a vehicle according to claim 2, wherein the data logger is configured to store time stamped information relating to at least one of the group consisting of:
activating the fog production unit,
a duration of the fog production operation, and
an amount of consumed liquid fogging medium.

4. A fog production unit for a vehicle according to claim 1, further comprising a disinfection container for holding disinfectant liquid.

5. A fog production unit for a vehicle according to claim 4, further comprising a mixer and wherein the disinfectant liquid is pumpable by a pump to the mixer, wherein the mixer is configured to receive the fogging medium and the disinfectant liquid and to provide a mixed liquid to the fogging nozzle.

6. A fog production unit for a vehicle according to claim 4, further comprising a dosing control for controlling the amount of disinfectant liquid in relation to the amount of fogging medium.

7. A fog production unit for a vehicle according to claim 1, further comprising an activation arrangement to activate the fog production unit.

8. A fog production unit for a vehicle according to claim 7, wherein the activation arrangement includes an activation button.

9. A fog production unit for a vehicle according to claim 8, wherein the activation button is implemented as a wireless button which is wirelessly connected to an associated receiver, such that, in operation, the receiver is configured to receive an activation signal from the wireless button.

10. A vehicle comprising:
a power unit for driving the vehicle,
a driver cabin comprising a control system for controlling acceleration, braking and steering of the vehicle,
a cargo space,
a pressurized air storage tank,
a compressor for supplying compressed air to the pressurized air storage tank, and
a fog production unit according to claim 1, and
wherein the fog production unit is connected to the pressurized air storage tank and
wherein the fogging nozzle of the fog production unit is mounted inside the cargo space.

11. A vehicle according to claim 10, wherein the compressor is mechanically driven by the power unit of the vehicle.

12. A vehicle according to claim 10, further comprising sensors for measuring environmental parameters within the cargo space and configured such that, in operation, the sensors provide sensor information to the data logger of the fog production unit.

13. A vehicle according to claim 12, wherein the environmental parameters comprise at least one of the following:
temperature of the cargo space,
humidity of the cargo space,
the open or closed state of the cargo space, and luminosity within the cargo space.

14. A vehicle according to claim 10, further comprising an activation arrangement to activate the fog production unit, and which is mounted in the driver cabin of the vehicle.

15. A method for installing a fog production unit according to claim 1 in a vehicle, comprising the steps of:
mounting the fog production unit in the vehicle, wherein the fogging nozzle is mounted within the cargo space;
connecting the air supply of the fog production unit to the pressurized air storage tank of the vehicle; and
mounting an activation arrangement to activate the fog production unit in the driver cabin.

16. A fog production unit for a vehicle according to claim 1, wherein:
the vehicle is a truck, and
the fluid delivery system includes one of:
a pump or
a venturi nozzle.
